(19) United States
(12) Patent Application Publication
(10) Pub. No.: US 2006/0211905 A1
(43) Pub. Date: Sep. 21, 2006

(54) Olefinic metathesis in the presence of phenolic compounds

(76) Inventors: Grant Stephen Forman, Fife (GB); Robert Paul Tooze, Fife (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/540,122

(22) PCT Filed: Nov. 3, 2003
(86) PCT No.: PCT/IB03/06502

§ 371 Date: Apr. 4, 2006

(30) Foreign Application Priority Data
Dec. 20, 2002 (GB) 0229770.3

(51) Int. Cl. C07C 11/00; C07C 6/00

(52) U.S. Cl. 585/645; 585/16

(57) ABSTRACT

According to the present invention there is provided a metathesis reaction between at least two olefinic compounds which are the same or different, each olefinic compound comprising a non-cyclic olefin or a compound which includes a non-cyclic olefinic moiety. The metathesis reaction is carried out in the presence of a Grubbs first generation catalyst and is characterised therein that it is carried out in the presence of a phenolic compound in the form of a phenol or a substituted phenol, which substituted phenol includes at least one hydroxyl and at least one further moiety other than H and OH attached to an arene ring.

### FIELD OF THE INVENTION

This invention relates to the enhancement of a metathesis reaction between at least two olefinic compounds which are the same or different.

### BACKGROUND ART

There is considerable interest regarding the formation of carbon-carbon bonds via olefin metathesis. The quest for highly active olefin metathesis catalysts has prompted considerable research efforts to develop olefin metathesis systems capable of tolerating a variety of functional groups. Ruthenium-based catalysts in particular have proven to be useful in catalysing olefin metathesis reactions, including cross metathesis (CM), ring-closing metathesis (RCM) and ring-opening metathesis polymerisation (ROMP) reactions.

Olefin metathesis refers to the metal-catalysed redistribution of carbon-carbon double bonds. CM can be described as a metathesis reaction between two non-cyclic olefins, which may be the same or different, for example:

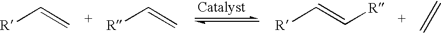

Where the olefins are the same, the reaction is known as self metathesis.

ROMP is a variant of olefin metathesis reactions wherein cyclic olefins produce polymers and co-polymers, for example:

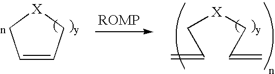

RCM represents a process in which an acyclic diene is cyclised to produce a cycloalkene, for example;

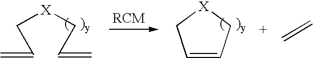

As indicated above metathesis reactions take place in the presence of a catalyst. Grubbs-type catalysts are ruthenium alkylidene complexes of the type shown below, and are highly active single component pre-catalysts for olefin metathesis reactions: 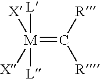
wherein:
M is ruthenium
X^{I} and X^{II} are independently an anionic ligand such as a halide;
R^{III} and R^{IIII} are independently hydrogen or an organyl group; and
L^{I} and L^{II} are independently a neutral electron donor ligand.

If each of L^{I} and L^{II} comprises a compound containing phosphorus wherein phosphorus is coordinated to M, then the catalyst is known as a Grubbs first generation catalyst, hereinafter referred to as Grubbs 1.

If one of L^{I} and L^{II} is not as defined for Grubbs 1, and especially if at least one of L^{I} or L^{II} comprises an N-heterocyclic carbene compound wherein the carbene carbon atom is coordinated to M, then the catalyst is known as a Grubbs second generation catalyst, hereinafter referred to as Grubbs 2. Such a carbene containing compound may for example comprise a substituted imidazolium ligand such as:

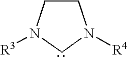

The production of bulk chemicals via ruthenium-catalysed metathesis, has the disadvantage that relatively high concentrations of costly ruthenium compounds are often required. This is often in excess of 1 mol % of substrate which destroys the economic viability of such processes. The use of suitably low catalyst concentrations is also confounded by the presence of impurities in typical olefinic feedstocks derived from primary processes such as naphtha cracking or the Fischer-Tropsch conversion of synthesis gas. During a study to identify possible catalyst poisons present in such feed streams it was surprisingly found that the addition of relatively low concentrations of phenol has a beneficial effect on catalyst performance in cross-metathesis, instead of the poisoning or retarding effect which was expected. Furthermore, it was most surprisingly found that the addition of phenol imparted to the catalyst a greater tolerance to feed impurities and known catalyst poisons. These impurities and poisons are described in more detail, later in this specification.

Thus in the presence of phenol it was found that CM reactions could be carried out at relatively low ruthenium concentrations, often giving good conversions of feedstock, high selectivity to desired products, and greater tolerance to feed impurities.

Such an effect has not been reported for this reaction before. In Macromolecules 2000, 33, 717-724 and in the Journal of Molecular Catalysis A. 2000, 160, 13-21, it was reported that the nature of the polymer formed by the ROMP reaction of two cyclic monomers was altered by running the reaction in chlorophenol as a solvent. This change was attributed to an unquantified increase in catalyst activity. The authors suggested that this was due to the phenolic solvent increasing the electrophilicity of the ruthenium centre.

However, the report of Grubbs and co-workers (J. Am. Chem. Soc. 1993, 115,9858-59) commented that whilst making the metal centre more electrophilic does lead to changes in the relative propagation rate of the ruthenium carbene centre in the polymerisation of norbornene, such complexes do not show improved activity for cross metathesis of pent-2-ene. This again suggests that the above effect is restricted to propagation rates of certain ROMP reactions and is not generally applicable to all types of metathesis reactions.

The effect reported for the present invention is significant at much lower levels of phenol addition, typically 0.02 mol l⁻¹ versus the bulk solvent employed in the above publication. Indeed examples shown below clearly indicate that there is no benefit to be gained by increasing the phenol concentration in this reaction.

The effect is not general for all metathesis reactions. The examples again show that phenol actually suppresses the rate of RCM. Neither is the effect uniform for all phenols and related compounds. Neither is it uniform for all metathesis catalysts, indeed the performance of the Grubbs 2 catalyst is diminished by the addition of phenol.

### DISCLOSURE OF THE INVENTION

According to the present invention there is provided a metathesis reaction between at least two olefinic compounds which are the same or different, each olefinic compound comprising a non-cyclic olefin or a compound which includes a non-cyclic olefinic moiety; the metathesis reaction being carried out in the presence of a catalyst of formula (I): 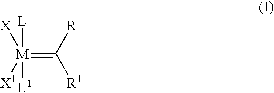
wherein:
M is ruthenium or osmium;
X and X¹ are independently selected from an anionic ligand;
R and R¹ are independently selected from H or an organyl group; and
L and L¹ are independently selected from any neutral electron donor ligand;
   and the metathesis reaction being characterised therein that it is carried out in the presence of a phenolic compound in the form of a phenol or a substituted phenol, which substituted phenol includes at least one hydroxyl and at least one further moiety other than H and OH attached to an arene ring of the phenol.

In this specification the term “a phenol” means a compound having one or more hydroxyl groups attached to a benzene ring or other arene ring.

It will be appreciated that the substituted phenol will include at least one hydroxyl group attached to the arene ring and at least one further moiety (other than H or OH) attached to the arene ring.

According to another aspect of the present invention there is provided the use of a phenolic compound in the form of a phenol or a substituted phenol which substituted phenol includes at least one hydroxyl and a further moiety other than H and OH attached to an arene ring, in a metathesis reaction between at least two olefinic compounds which are the same or different, each olefinic compound comprising a non-cyclic olefin or a compound which includes a non-cyclic olefinic moiety, and the metathesis reaction being carried out in the presence of a catalyst of formula (I) as defined above.

The metathesis reaction may be enhanced by the phenolic compound and it may be enhanced in at least one of the following ways:
i) increased lifetime of the catalyst;
ii) increased resistance of the catalyst to olefin feed impurities, including oxygen-containing compounds;
iii) increased selectivity of the metathesis reaction in respect of at least one of the following aspects:
   a. reduced isomerisation of the starting olefinic compound(s);
   b. reduced formation of secondary metathesis product(s);
iv) increase in the yield of the metathesis product(s);
v) increase in the rate of the reaction; and
vi) use of lower catalyst concentrations possible.

It will be appreciated that the effects are observed compared to the same reaction under the same condition but in the absence of the phenolic compound.

It has been found (from the examples below) that the less expensive Grubbs 1 catalysts with phenolic compounds provide results comparable to that obtained with the more expensive Grubbs 2 catalyst.

Preferably the metathesis reaction between the at least two olefinic compounds does not result in opening of a ring structure which may form part of at least one of the olefinic compounds.

Preferably the metathesis reaction produces a product which does not include a cyclic moiety formed by the metathesis reaction. It will be appreciated that a starting olefinic compound may include a cyclic olefin, but preferably the metathesis reaction does not cause a cyclic moiety to form. Preferably one or more non-cyclic olefins are produced by the metathesis reaction.

The Starting Olefinic Compound(s)

Preferably the metathesis reaction is between at least two non-cyclic olefins which are the same or different. Preferably at least one, but preferably all of the at least two non-cyclic olefins comprise an olefin with a single double bond. Preferably at least one, but preferably all of the at least two non-cyclic olefins comprise a 1-alkene. The 1-alkene may comprise a non-branched alkene for example 1-octene or 1-heptene.

In one embodiment of the invention the metathesis reaction may be between at least two different non-cyclic olefins, preferably between only two different non-cyclic olefins. In a further embodiment of the invention the one non-cyclic olefin may comprise ethylene and the second non-cyclic olefin may comprise an internal olefin, preferably an internal olefin with a single double bond.

In an alternative embodiment of the invention the metathesis reaction may be between two non-cyclic olefins which are the same. The non-cyclic olefin may be an olefin with a single double bond, preferably a 1-alkene, preferably a non-branched 1-alkene (for example 1-octene or 1-heptene).

In an alternative embodiment of the invention the metathesis reaction may be between at least two non-cyclic olefins of which at least one is contained in a feedstock derived from a Fischer-Tropsch reaction. Such a feedstock is often very complex and may contain non-branched and branched 1-alkenes, non-branched and branched internal alkenes, as well as alkenes, aromatics and various oxygenated impurities.

The said feedstock containing the non-cyclic olefin may further contain at least one impurity selected from the group consisting of a carbonyl containing compound, an alcohol, an aromatic compound, a diene, a triene, an alkyne and an aldehyde. These impurities may act as poisons which interfere with the catalytic activity of the catalysts of this invention, in the absence of a phenolic compound. The interference with the catalyst may be in the form of retardation of the conversion of the starting olefin, or by permanently deactivating the catalyst.

The Catalyst

With reference to formula (I) M is preferably Ru.

X and X¹ may be independently selected from the group consisting of hydrogen; halide; and a compound selected from the group consisting of C₁-C₂₀ alkyl; aryl; C₁-C₂₀ alkoxide; aryloxide; C₃-C₂₀ alkyldiketonate; aryldiketonate; C₁-C₂₀ carboxylate; arylsulfonate; C₁-C₂₀ alkylsulfonate; C₁-C₂₀ alkylthiol; aryl thiol; C₁-C₂₀ alkylsulfonyl; and C₁-C₂₀ alkylsulfinyl, the compound being optionally substituted with one or more other moieties selected from the group consisting of C₁-C₁₀ alkyl; C₁-C₁₀ alkoxy; aryl and halide. Preferably X and X¹ are each independently selected from the group consisting of halide; CF₃CO₂; CH₃CO_{2,}; CFH₂CO₂; (CH₃)₃CO; (CF₃)₂(CH₃)CO; (CF₃)(CH₃)₂CO; PhO; MeO; EtO; tosylate; mesylate; and trifluoromethanesulfonate. Preferably X and X¹ are each independently selected from halide. Preferably X and X¹ are each chloride.

R and R¹ are each independently selected from the group consisting of hydrogen and an organyl selected from the group consisting of C₁-C₂₀ alkyl; C₂-C₂₀ alkenyl; C₂-C₂₀ alkynyl; aryl; C₁-C₂₀ carboxylate; C₁-C₂₀ alkoxy; C₂-C₂₀ alkenyloxy; C₂-C₂₀ alkynyloxy; aryloxy; C₂-C₂₀ alkoxycarbonyl; C₁-C₂₀ alkylthiol; aryl thiol; C₁-C₂₀ alkylsulfonyl and C₁-C₂₀ alkylsulfinyl, the organyl being optionally substituted with one or more moieties selected from the group consisting of C₁-C₁₀ alkyl; C₁-C₁₀ alkoxy; aryl; and a functional group selected from the group consisting of hydroxyl; thiol; thioether; ketone; aldehyde; ester; ether; amine; imine; amide; nitro; carboxylic acid; disulfide; carbonate; isocyanate; carbodiimide; carboalkoxy; carbamate; and halogen. Preferably R is hydrogen and R¹ is phenyl or vinyl, optionally substituted with one or more moieties selected from the group consisting of C₁-C₅ alkyl; C₁-C₅ alkoxy; phenyl; and a functional group selected from the group consisting of hydroxyl; thiol; thioether; ketone; aldehyde; ester; ether; amine; imine; amide; nitro; carboxylic acid; disulfide; carbonate; isocyanate; carbodiimide; carboalkoxy; carbamate; and halogen. Preferably R is H and R¹ is phenyl or —C═C(CH₃)₂.

L and L¹ are each independently selected from the group consisting of phosphine, sulfonated phosphine, phosphite, phosphinite, phosphonite, arsine, stibine, amine, amide, imine, nitrosyl and pyridine. Preferably L and L¹ are each a compound containing phosphorus, preferably with the phosphorus atom co-ordinated to M. Preferably L and L¹ are independently a phosphine preferably a phosphine of the formula PR³R⁴R⁵, wherein R³, R⁴ and R⁵ are each independently aryl, C₁-C₁₀ alkyl or cycloalkyl. Preferably L and L¹ are each independently selected from the group consisting of —P(cyclohexyl)₃; —P(cyclopentyl)₃; —P(isopropyl)₃; and —P(phenyl)₃. Preferably L and L¹ may be a phosphacycloalkane or phosphabicycloalkane such as eicosyl phoban. Preferably L and L¹ are the same.

It is foreseen that at least some of X, X¹, R, R¹, L and L¹ may be bound to each other.

In one embodiment of the invention the catalyst of formula (I) may comprise a compound of formula (II): 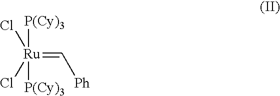
wherein Cy is cyclohexyl.

In one embodiment of the invention the catalyst may be prepared prior to addition of the catalyst to the metathesis reaction. In an alternative embodiment of the invention the catalyst may be prepared in situ in the reaction medium of the metathesis reaction.

The Phenolic Compound

In one embodiment of the invention the phenolic compound may comprise a phenol, including phenol.

In another embodiment of the invention the phenolic compound may comprise a substituted phenol which includes at least one hydroxyl and at least one further moiety other than H and OH attached to an arene ring. The substituted phenol may comprise a substituted phenol of the formula: 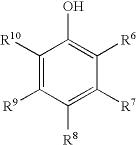
wherein R⁶, R⁷, R⁸, R⁹ and R¹⁰ are each independently selected from H and any suitable moiety and at least one of R⁶, R⁷, R⁸, R⁹ and R¹⁰, is not H and OH. The moiety may comprise (but is not limited to) halide; oxy organyl (including alkoxy), benzoate, carboxylate, alkyl (for example Me, Et, ^{t}Bu), phenol, alkylthio, aryl, alkylsulfonate, tosylate, mesylate, cycloalkyl, arylsulfonate, alkylketone, alkyldiketone, alkylsulfinyl, phosphine, phosphinate, phosphate, hydroxyl, alkoxycarbonyl, amino, nitro, haloalkyl, nitrile.

In another embodiment of the invention the phenolic compound may comprise an optionally substituted polyaromatic phenol e.g. naphthol, including 1-naphthol and 2-naphthol.

In another embodiment of the invention the phenolic compound may comprise an optionally substituted bis-phenol eg. hydroquinone or catechol.

The substituted phenol may be selected from, but is not restricted to, the group consisting of cresol; ethyl phenol (including 4-Et-phenol); methoxyphenol (including 4-OMe-phenol); cyanophenol (including 4-CN-phenol); chlorophenol (including 4-chlorophenol)fluorophenol (including 4-fluorophenol), iodophenol (including 4-iodophenol)trifluoromethylphenol (including 4-CF₃-phenol); and 2,6-di-tert-butyl-4-methylphenol (BHT).

The concentration of the phenolic compound may be varied as required. In one embodiment of the invention the molar ratio of phenolic compound to catalyst may be from 1 to 5000 molar equivalents of phenolic compound to ruthenium or osmium, preferably in the range of 200 to 1000 molar equivalents.

The concentration of catalyst is preferably 1 ppm to 500 ppm, preferably 20 ppm to 200 ppm preferably about 100 ppm.

The phenolic compound may be used in combination with a solvent.

The phenolic compound maybe incorporated onto a solid support

The present invention also relates to an olefin produced by the process or use set out above.

The invention will now be further described by means of the following non-limiting examples.

### EXAMPLES

General Experimental Procedure

Unless otherwise stated, the general experimental procedure for the metathesis reactions of the examples were performed in a 250 ml round-bottom flask. 1-Octene was purified by passing it through an alumina column and it was stored over alumina in the absence of light. The stock 1-octene solution was degassed with N₂ 30 minutes before use, and 20 ml was transferred to a 250 ml flask containing the phenol additive. [Unless stated otherwise the control contained no phenol additive]. The solution was degassed at room temperature for 30 minutes before being heated to 50° C. A Grubbs 1^{st} generation catalyst of formula II (referred to as G1) was weighed into a custom-made miniature aluminium weighing tray. An amount of 11.2 mg of the catalyst provided a Ru concentration of 100 ppm. The weighing tray containing the catalyst was added, and the reaction stirred for 6 hours. Samples were taken thereafter every 10, 30, 60, 120, 180, 240 and 360 minutes and analysed by GC-FID. Conversions are reported as the molar % 1-octene which was converted to the desired 7-tetradecene product.

Unless stated otherwise the reference to equivalents of a compound refers to molar equivalents of the compound to the catalyst. For example 500 eq of phenol refers to a phenol to catalyst molar ratio of 500:1.

### Example 1

### The Phenol Enhancement Effect

The general experimental procedure was followed without addition of any phenol and with the addition of 500 eq of phenol.

An increased conversion of 1-octene to the desired 7-tetradecene product was observed for the metathesis of 1-octene when 500 equivalents of phenol was added to the reaction mixture (Table 1) compared to the reaction where no phenol was added. Under these conditions, no detectable (by GC) amounts of isomerised octene or secondary metathesis products (SMP's) were observed. Furthermore, the catalyst was active even after four hours, in marked contrast to the control experiment where no phenol was added. In an effort to verify the results obtained above, the reaction was repeated. These results indicate that the reaction can be readily reproduced.

TABLE 1
Metathesis of 1-octene with G1 (no phenol) vs G1 + phenol (500 eq) at
50° C.

| | Molar % Conversion of 1-Octene |
|---|---|
| Conditions | After 3 h |
| G1 with no phenol added | 26.5% |
| G1 + 500 eq phenol | 82.0% |
| G1 + 500 eq phenol (repeated) | 86.6% |

FIG. 1 below shows an improved reaction rate where G1 is used in combination with phenol compared to where G1 without phenol is used.

[image]

### Example 2

### Effect of Phenol Concentration

The 1-octene metathesis reactions were performed following the general experimental procedure and using 200, 500 and 1000 equivalents of phenol, respectively. The results are shown in Table 2. Under these conditions, optimum performance is achievable with either 500 or 1000 equivalents of phenol.

TABLE 2
Metathesis of 1-octene with G1 + phenol (200, 500 and 1000eq).

| | Molar % Conversion of 1-Octene |
|---|---|
| Conditions | After 3 h |
| G1 with no phenol added | 26% |
| G1 + 200 eq phenol | 63% |
| G1 + 500 eq phenol | 82% |
| G1 + 1000 eq phenol | 77% |

### Example 3

### Effect of Substitution on Phenol

In an effort to asses the effect of substitution on the benzene ring of the phenol on the formation of 7-tetradecene in 1-octene metathesis, 500 equivalents of compounds 1-6 were added to the reaction mixture, following the general experimental procedure. The results are summarized in Table 3.

TABLE 3
Metathesis of 1-octene with substituted phenols

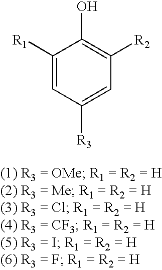

| | Molar % Conversion of 1- |
|---|---|
| Conditions | Octene After 3 h |
| G1 with no phenol added | 26.5% |
| G1 + 500 eq 4-Cl-phenol (Compound 3) | 58.2% |
| G1 + 500 eq 4-CF₃-phenol (Compound 4) | 64.8% |
| G1 + 500 eq 4-I-phenol (Compound 5) | 71.0% |
| G1 + 500 eq 4-OMe-phenol (Compound 1) | 71.8% |
| G1 + 500 eq phenol | 82.0% |
| G1 + 500 eq 4-F-phenol (Compound 6) | 86.0% |
| G1 + 500 eq 4-Me-phenol (cresol) | 91.1% |
| (Compound 2) | |

These results show that the addition of 500 equivalents of p-cresol (compound 2) afforded very similar (but slightly better) yields compared to those obtained with phenol. 4-Methoxyphenol (compound 1) gave slightly lower conversions than phenol and p-cresol, Electron-withdrawing groups and electronegative substituents present on the phenol moiety (compounds 3-5) gave lower conversions compared with those results obtained with phenol, but still better than the control without phenol. Compound 6 also has electronegative substituents but gave better results than phenol.

Other additives were also tested, namely BHT (2,6 di-tert-butyl-4-methyl phenol)naphthols, hydroquinone and catechol while following the general experimental procedure. Results are shown in Table 4.

TABLE 4
Metathesis of 1-octene with other phenols.

| | Molar % Conversion of 1-Octene |
|---|---|
| Conditions | After 3 h |
| G1 with no phenol added | 27% |
| G1 + 500 eq hydroquinone | 55% |
| G1 + 500 eq 2-napthol | 65% |
| G1 + 500 eq catechol | 76% |
| G1 + 500 eq 4-1-napthol | 77% |
| G1 + 500 eq BHT | 79% |
| G1 + 500 eq phenol | 82% |

The results show that the conversion of 1-octene to 7-tetradecene is increased by the addition of BHT, napthols, hydroquinone and catechol relative to the control G1 metathesis experiment using G1 with no phenol added.

### Example 4

### Tolerance to Feed Impurities

1-Alkenes derived from Fisher-Tropsch product streams may contain various impurities that are capable of retarding or even deactivating olefin metathesis catalysts. In an effort to discover whether phenolic compounds could be used in metathesis reactions of such alkene feedstocks (for example to improve catalyst lifetime, catalyst robustness and tolerance to feed impurities) the following experimental work was performed.

a) Hexanone as Feed Impurity

2-Hexanone was chosen as an initial model poison (impurity) as previous studies have shown that it gives rise to low metathesis yields and significant amounts of isomerised alkenes during cross-metathesis reactions (for example, metathesis of 1-octene at Ru:100 ppm (G1), 50° C. with 100 molar eq (relative to Ru) 2-hexanone provides only 15% 7-tetradecene, 9% 2-octene after 6 hours). Thus, G1 (100 ppm) was added to a solution of 2-hexanone (100 molar eq wrt Ru) and phenol (500 eq) in 1-octene at 50° C. and following the general experimental procedure. The results are summarized in Table 5.

TABLE 5
Metathesis of 1-octene with 100 eq of 2-hexanone and 500 eq of
phenol.

| | Molar % Conversion of 1-Octene |
|---|---|
| Conditions | After 3 h |
| G1 with no phenol added | 27% |
| G1 + 100 eq 2-hexanone (no phenol) | 9% |
| G1 + 500 eq phenol + 100 eq 2- | 87% |
| hexanone | |
| G1 + 500 eq phenol (no 2-hexanone) | 82% |

Under these conditions the conversion of 1-octene to 7-tetradecene was almost identical to the control experiment, where no 2-hexanone is added. Most significantly, no detectable quantities of 2-octene were observed during the reaction.

b) Fischer-Tropsch Feed Simulation

Efforts were made to test the G1 and phenol combination against a crudely simulated Fisher-Tropsch derived feedstock. An impure feedstock was prepared to include a typical range of impurities. The following compounds were added to purified Aldrich 1-octene;

Acetone (100 eq/eq Ru catalyst)

Ethanol (125 eq/eq Ru catalyst)

Toluene (70 eq/eq Ru catalyst)

Cycloheptatriene (70 eq/eq Ru catalyst)

Cyclooctadiene (60 eq/eq Ru catalyst)

1-Hexanol (60 eq/eq Ru catalyst)

2-Hexanone (60 eq/eq Ru catalyst)

Hexanal (60 eq/eq Ru catalyst)

3-Hexyne (70 eq/eq Ru catalyst)

Phenol (500 eq) was then added, followed by G1 (Ru:100 ppm) and the general experimental procedure was followed to compare 1-octene conversion against the control experiment in which no phenol was added to the impure feed. The results are shown in Table 6. These results show that upon addition G1 to 500 equivalents of phenol in the impure 1-octene feed, a greater conversion is observed compared with the control experiment in which no phenol is added (32.8% conversion vs 6.1% conversion after 3 hours).

TABLE 6
1-Octene conversion (impure feed) with 500 eq of phenol.

| | Molar % Conversion of 1-Octene |
|---|---|
| Conditions | After 3 h |
| G1 with no phenol added | 6.1% |
| G1 + 500 eq phenol | 32.8% |

c) Untreated 1-octene

As stated under the general experimental procedure 1-octene (in this case commercial Aldrich 1-octene) is typically percolated through an alumina column and stored over a bed of alumina and under an inert atmosphere before use. This treatment destroys any traces of peroxides which might form in the feed in the presence of air. The general experimental procedure was again followed, but this time using untreated 1-octene. The results summarized in Table 7 below show that 1-octene cross-metathesis is possible using untreated 1-octene, giving 50% conversion after 3 hours. This result compares favourably to the control experiment, using unpurified 1-octene with no phenol, which gave rise to only 8.5% conversion to desired 7-tetradecene product after 3 hours.

TABLE 7
Metathesis of 1-octene (untreated feed) with 500 eq of Phenol.

| | Molar % Conversion of 1-Octene |
|---|---|
| Conditions | After 3 h |
| G1 with no phenol added | 8.5% |
| G1 + 500 eq phenol | 50.0% |

### Example 5

### Effect of Phenol on Metathesis of Olefins Derived from Fischer-Tropsch Product Streams

The general experimental procedure was followed.

a) FT Derived Feed A

A Fischer-Tropsch (FT) derived C₇ feed with the following composition was employed:

| | |
|---|---|
| Linear 1-alkene | 86% |
| Linear internal alkene | 1-1.5% |
| Branched alkene (incl. internal) | 5-7% |
| Cyclic alkene | 1-2% |
| Diene | 1% |
| Oxygenate | <100 ppm |
| Paraffins, aromatics & other | 5-7% |

Reactions were carried out at 50° C. and at 100 ppm Ru. 500 Equivalents of phenol was added relative to Ru. Conversions of 1-heptene to the desired product (6-dodecene) are shown in Table 8 below. In the control no phenol was added. It is clear that the addition of phenol improved product yields significantly despite the presence of trace poisons.

TABLE 8
Effect of phenol addition on Fischer-Tropsch derived C7 washed feed

| | Molar % Conversion of 1-Heptene |
|---|---|
| Conditions | After 3 h |
| G1 with no phenol added | 32% |
| G1 + 500 eq phenol | 60% |

b) FT Derived Feed B

A Fischer-Tropsch derived C₇ feed of the following composition was employed:

| | |
|---|---|
| Linear 1-alkene | 83% |
| Linear internal alkene | 1-2% |
| Branched alkene (incl. internal) | 4-5% |
| Cyclic alkene | 1-2% |
| Diene | 1-3% |
| Oxygenate | 4-5% |
| Paraffins, aromatics & other | 4-6% |

Reactions were carried out at 50° C. and at 100 ppm Ru. Efforts were made to investigate the effect of increasing phenol concentration, and the results are shown in Table 9 below. It is evident that addition of phenol does improve reaction yields, and that addition of excess phenol up to a certain level (˜1000 eq) further improves catalyst performance.

TABLE 9
The effect of phenol as additive on unwashed feed derived from a
Fischer-Tropsch process.

| | Molar % Conversion of 1-Heptene |
|---|---|
| Conditions | After 3 h |
| G1 with no phenol added | 7% |
| G1 + 500 eq phenol | 12% |
| G1 + 1000 eq phenol | 23% |

### Example 6

### Effect of Phenol on Ethenolysis

Experimental Procedure:

Reactions were carried out in a 50 ml Parr autoclave fitted with a stirrer, a diptube for gas entrainment, and an outlet (vent) line. The reactor was flushed with argon, sealed and flushed with ethylene. 16 ml of 2-Octene feed was transferred via syringe to an ethylene flushed sample bomb and introduced to the reactor under a few bar of ethylene pressure. The ethylene is extremely soluble in the 2-octene mixtures, thus the mixture was stirred under ethylene pressure until no further pressure drop was noted. The catalyst was weighed into a sample tube, dissolved in 8 ml dichloromethane, transferred via syringe to the sample bomb, and introduced to the reactor under ethylene pressure so as to reach the desired reaction pressure in the autoclave. As ethylene dissolved in the mixture and reactor pressure dropped, the pressure was maintained by opening the gas inlet line. Once the desired pressure could be maintained with no further pressure drop, the system was closed and left to stir for 15 h. Samples were taken at the end of the run and analysed by GC-FID.

Experiments were carried out at 25° C. and 50 ppm Ru using G1. The substrates employed were:

1) 98% 2-octene, containing 80% cis and 20% trans 2-octene.

2) 98% 2-octene, containing 99% trans-2-octene

Results are summarised in Table 10:

TABLE 10
Ethenolysis of 2-octene to 1-heptene using G1

| | MOLAR % | |
|---|---|---|
| | CONVERSION | |
| | OF 2-OCTENE | SELECTIVITY |
| 2-OCTENE (4:1 CIS:TRANS) | 87% | 95% |
| 2-OCTENE (4:1 CIS:TRANS) + | 92% | 93% |
| PhOH | | |
| 2-OCTENE (99% TRANS) | 73% | 92% |
| 2-OCTENE (99% TRANS) + PhOH | 84% | 94% |

Selectivity refers to the amount of heptenes formed relative to the total products formed (including slef and self secondary metathesis products).

It is evident that significantly higher yields of the desired α-olefin product were obtained with addition of phenol. When considering selectivities, these were approximately similar with and without phenol: However, phenol addition gave less isomerisation of the 1-alkene product as a side reaction but slightly more formation of 6-dodecene, the product of self-metathesis of 2-octene. The use of phenol would, therefore, be preferred.

### Example 7

### Effect of Phenol where Catalyst is Prepared In Situ

The in-situ preparation of a Grubbs-type catalyst in a metathesis reaction medium is known. This catalyst system was reported by Nubel and Hunt in J. Molec. Catal. A, 1999, 145, 323-327 and U.S. Pat. No. 6,159,890 and involves in situ generation of the active catalyst by the addition of RuCl₃, a phosphine and 1,4-butynediol diacetate (BDD) to the alkene substrate in the presence of a hydrogen sparge.

Experimental Procedure:

Reactions were carried out in a 100 ml three-necked flask fitted with a reflux condenser, thermometer and septum. The reflux condenser was connected to a cooling bath set at 5° C. to ensure a constant flow of chilled water through the jacket, thereby preventing loss of octene. The top of the condenser was connected to a bubbler in order to monitor liquid losses and gas emissions. The thermometer was positioned below the level of the reaction solution to ensure correct temperature monitoring. A needle inserted through the septum and connected to a gas supply via a needle valve was used to ensure a slow and steady stream of argon or hydrogen bubbles through the reaction solution. Argon was used during the initial phase to purge the system. After all the reagents were added, the gas flow was changed to hydrogen and the reaction was heated at the desired temperature by means of a preheated oil bath, and stirred by means of a magnetic stirrer bar. Samples were taken at regular intervals via syringe through the septum and analysed by GC-FID. Unless otherwise stated, 20 ml of octene was employed in all experiments, and catalyst, solvent and additive amounts were calculated relative to this. 0.5 ml of octadecane was used as internal standard. Results are reported as the molar % yield of tetradecene.

The effect of phenol addition was tested for two different phosphine ligands namely P(cyclohexyl)₃ [PCy₃] and eicosyl phoban (EP) using 100 ppm Ru and 500 molar eq phenol relative to Ru. It is evident from Table 11 below that the phenol effect significantly enhances the performance of this in situ system. In addition it is clear that this enhancement is also observed when employing alternative phosphine ligands to the standard tricyclohexylphosphine ligand.

TABLE 11
In situ system: PCy₃ and EP with and without phenol at 100 ppm Ru

| | Molar % Conversion of |
|---|---|
| Conditions | 1-Octene After 3 h |
| 100 ppm Ru, 80 degrees | 30% |
| Ru:phenol:PCy₃:BDD = 1:0:2.5:10 | |
| 100 ppm Ru, 80 degrees | 67% |
| Ru:phenol:PCy₃:BDD = 1:500:2.5:10 | |
| 100 ppm Ru, 80 degrees | 22% |
| Ru:phenol:EP:BDD = 1:0:2.5:10 | |
| 100 ppm Ru, 80 degrees | 45% |
| Ru:phenol:EP:BDD = 1:500:2.5:10 | |

### Comparative Example 8

### Effect of Phenol with a Grubbs 2^{nd} Generation Catalyst (G2)

The Grubbs 2^{nd} generation catalyst (G2) used in the present example was the compound:

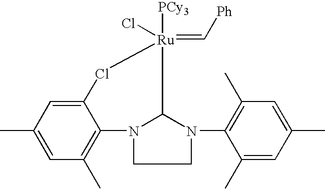

This catalyst (G2) affords rapid but unselective cross-metathesis of 1-alkenes compared to G1. The comparative catalyst performance of G2 (with and without phenol) to G1 and phenol (500 equivalents per metal) was investigated for the metathesis of 1-octene following the general experimental procedure. The results are summarised in Table 12.

These results show that after 3 hours conversions obtained using G2 catalyst in the presence of phenol is significantly lower than that afforded by G2 alone. It also clearly shows that the conversion using G1 plus phenol is similar to that using G2 alone. Additionally noticeable amounts of 2-octene and secondary metathesis products (SMP) were observed when G2 was employed in the absence of phenol. These SMP compounds are not observed when using G1 with added phenol.

TABLE 12
Conversion of 1-octene with G2, (without phenol), G1 + 500 eq
phenol and G2 + 500 eq phenol.

| | Molar % Conversion | |
|---|---|---|
| Conditions | of 1-Octene after 3 h | Selectivity |
| G1 with no phenol | 26.5% | 96% |
| added | | |
| G2 with no phenol | 91.2% | 56% |
| added | | |
| G2 + 500 eq phenol | 68.9% | 61% |
| G1 + 500 eq phenol | 82.0% | 98% |

Selectivity is expressed as the amount of 7-tetradecene formed relative to the total products (including isomerised 1-octene and secondary metathesis products such as tridecene, dodecene etc.).

### Comparative Example 9

### Effect of Phenol on Ring Closing Metathesis

In this investigation diethyl diallyl malonate was used as a starting material and was converted to the ring-closed product using G1.

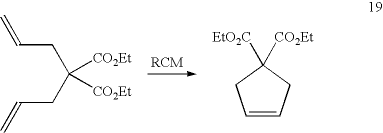

G1 catalyst was weighed into a custom-made miniature aluminium weighing tray. An amount of 11.2 mg of the catalyst provided a Ru concentration of 100 ppm. The weighing tray containing the catalyst was added to a solution of diethyl diallyl malonate (13.5 ml, 4000 eq) dissolved in 15 ml CH₂Cl₂ at 50° C., and the reaction stirred for 6 hours. GC samples were taken thereafter every 10, 30, 60, 120, 180, 240 and 360 minutes.

An identical reaction was set-up in which 500 equivalents of phenol was added to a solution of diethyl diallyl malonate (13.5 ml, 4000 eq) dissolved in 15 ml CH₂Cl₂ at 50 degrees before G1 was added. The results are summarized in Table 13. These results show that the addition of phenol retards RCM of diethyl diallyl malonate.

TABLE 13
Ring-Closing metathesis of diethyl diallyl malonate using G1
and G1 + 500 eq phenol.

| | Molar % conversion of 1-diethyl |
|---|---|
| Conditions | diallyl malonate after 3 h |
| G1 with no phenol added | 39.0% |
| G1 + 500 eq phenol | 25.4% |

### Comparative Example 10

### Non-Phenolic Additives

Non-phenolic additives that are structurally related to phenol were used in a metathesis reaction set out in the general experimental procedure. That is the phenol additive was in each case replaced with a non-phenolic additive using 500 molar equivalents of additive to ruthenium catalyst.

The results indicate that these additives do not exhibit the beneficial effect in metathesis observed for the phenolic compounds reported above.

TABLE 14
Metathesis of 1-octene with non-phenolic additives

| | Molar % Conversion of 1-Octene |
|---|---|
| Conditions | After 3 h |
| G1 with no phenol added | 26.5% |
| G1 + 500 eq aniline | 0% |
| G1 + 500 eq thiophenol | 0% |
| G1 + 500 eq cyclohexanol | 13.4% |
| G1 + 500 eq anisole | 17.8% |
| G1 + 500 eq phenol | 82.0% |

### Example 11

### Metathesis Using a Solvent

While in many instances metathesis is carried out “neat” with no additional solvent, solvents may be added, particularly if the olefin reagents are solids. Table 15 below shows that the enhancing effect of phenol addition is also observed when metathesis is carried out with a solvent.

Reactions were performed in a 250 ml round-bottom flask. 1-Octene was purified by passing it through an alumina column and it was stored over alumina in the absence of light. The stock 1-octene solution was degassed with N₂ 30 minutes before use, and 20 ml was transferred to a 250 ml flask containing 20 ml of the solvent and the phenol additive. [Unless stated otherwise the control contained no phenol additive]. The solution was degassed at room temperature for 30 minutes before being heated to 50° C. A Grubbs 1^{st} generation catalyst of formula II (referred to as G1) was weighed into a custom-made miniature aluminium weighing tray. An amount of 11.2 mg of the catalyst provided a Ru concentration of 100 ppm. The weighing tray containing the catalyst was added, and the reaction stirred for 6 hours. Samples were taken thereafter every 10, 30, 60, 120, 180, 240 and 360 minutes and analysed by GC-FID. Conversions are reported as the molar % 1-octene which was converted to the desired 7-tetradecene product.

TABLE 15
Metathesis of 1-octene in solvent with G1 (no phenol) and
G1 + (500 eq) phenol.

| | Molar % Conversion | Molar % Conversion |
|---|---|---|
| | of 1-Octene after 3 h | of 1-Octene after 3 h |
| | Using G1 with no | Using G1 and phenol |
| Solvent | phenol | (500 eq) |
| Toluene | 13% | 62% |
| Decane | 19% | 76% |
| Cyclohexane | 24% | 77% |
| Diphenylether | 29% | 77% |

### Example 12

### Generality to 1^{st} Generation Catalysts

The general experimental procedure was followed except that G1 was replaced with other Grubbs 1^{st} generation catalysts. 20 mL of 1-octene, 100 ppm of catalyst and 500 eq phenol were used and the reactions were run at 50° C.

Results are shown in table 16 below. It is evident that the enhancing effect of phenol is observed for different types of first generation Grubbs catalysts, including those with different neutral ligands and different anionic ligands.

TABLE 16
Metathesis of 1-octene using different catalysts with and without phenol.

| | Molar % | |
|---|---|---|
| | Conversion of | Molar % |
| | 1-Octene | Conversion of |
| | after 3 h without | 1-Octene after 3 h |
| Catalyst | added phenol | with added phenol |
| RuCl₂(PCy₃)(═CHPh) | 26% | 83% |
| RuCl₂(PCp₃)(═CHPh) | 2% | 24% |
| RuBr₂(PCy₃)(═CHPh) | 15% | 52% |
| RuCl₂(PCy₃)(py)₂(═CHPh) | 3% | 9% |

Where:
Py = pyridine
Ph = phenyl
Cp = cyclopentyl

### Example 13

### Performing the Reaction at Low Catalyst Loadings

The general experimental procedure was followed, with the addition of catalyst and phenol as per the levels stipulated in table 17. The absolute phenol concentration in the reaction mixture was kept constant in order to determine the effect of ruthenium concentration.

The combination of G1 with phenol allows reactions to be performed at low catalyst loadings. Results are shown in table 17 below. It is evident that the enhancing effect of phenol is observed at catalyst loadings as low as 25 ppm Ru. The results show that the positive enhancing effect of phenol is applicable for even very low catalyst loadings.

TABLE 17

| | Molar % Conversion of | Molar % Conversion of |
|---|---|---|
| Concentration of | 1-Octene after 3 h | 1-Octene after 3 h with |
| catalyst (G1) | without added phenol | added phenol |
| 100 ppm | 26% | 83% |
| 50 ppm | 17% | 78% |
| 25 ppm | 7% | 81% |

1. A metathesis reaction between at least two olefinic compounds which are the same or different, each olefinic compound comprising a non-cyclic olefin or a compound which includes a non-cyclic olefinic moiety; the metathesis reaction being carried out in the presence of a catalyst of formula (I): 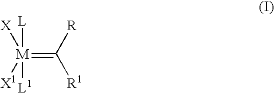
wherein:
M is ruthenium or osmium;
X and X¹ are independently selected from an anionic ligand;
R and R¹ are independently selected from H or an organyl group; and
L and L¹ are independently selected from any neutral electron donor ligand;
and the metathesis reaction being characterised therein that it is carried out in the presence of a phenolic compound in the form of a phenol or a substituted phenol, which substituted phenol includes at least one hydroxyl and at least one further moiety other than H and OH attached to an arene ring.

2. The metathesis reaction as claimed in claim 1, wherein a product is produced which does not include a cyclic moiety formed by the metathesis reaction.

3. The metathesis reaction as claimed in either one of claims 1 or 2, wherein the metathesis reaction is between two non-cyclic olefins which are the same or different.

4. The metathesis reaction as claimed in claim 3 wherein each of the non-cyclic olefins comprises an olefin with a single double bond.

5. The metathesis reaction as claimed in claim 4, wherein the metathesis reaction is between ethylene and an internal non-cyclic olefin.

6. The metathesis reaction as claimed in claim 4, wherein the metathesis reaction is between two non-cyclic olefins which are the same.

7. The metathesis reaction as claimed in claim 6, wherein the non-cyclic olefins are both a non-branched 1-alkene.

8. The metathesis reaction as claimed in claim 3, wherein the metathesis reaction is between at least two non-cyclic olefins of which at least one is contained in a feedstock derived from a Fischer-Tropsch reaction.

9. The metathesis reaction as claimed in claim 8, wherein the feedstock contains at least one impurity selected from the group consisting of a carbonyl containing compound, an alcohol, an aromatic compound, a diene, a triene, an alkyne and an aldehyde.

10. The metathesis reaction as claimed in any of the preceding claims, wherein M in formula (I) is ruthenium.

11. The metathesis reaction as claimed in any of the preceding claims, wherein X and X¹ are independently selected from the group consisting of hydrogen; halide; and a compound selected from the group consisting of C₁-C₂₀ alkyl; aryl; C₁-C₂₀ alkoxide; aryloxide; C₃-C₂₀ alkyldiketonate; aryldiketonate; C₁-C₂₀ carboxylate; arylsulfonate; C₁-C₂₀ alkylsulfonate; C₁-C₂₀ alkylthiol; aryl thiol; C₁-C₂₀ alkylsulfonyl; and C₁-C₂₀ alkylsulfinyl, the compound being optionally substituted with one or more other moieties selected from the group consisting of C₁-C₁₀ alkyl; C₁-C₁₀ alkoxy; aryl and halide.

12. The metathesis reaction as claimed in any of the preceding claims, wherein X and X¹ are each chloride.

13. The metathesis reaction as claimed in any of the preceding claims, wherein R and R¹ are each independently selected from the group consisting of hydrogen and an organyl selected from the group consisting of C₁-C₂₀ alkyl; C₂-C₂₀ alkenyl; C₂-C₂₀ alkynyl; aryl; C₁-C₂₀ carboxylate; C₁-C₂₀ alkoxy; C₂-C₂₀ alkenyloxy; C₂-C₂₀ alkynyloxy; aryloxy; C₂-C₂₀ alkoxycarbonyl; C₁-C₂₀ alkylthiol; aryl thiol; C₁-C₂₀ alkylsulfonyl and C₁-C₂₀ alkylsulfinyl, the organyl being optionally substituted with one or more moieties selected from the group consisting of C₁-C₁₀ alkyl; C₁-C₁₀ alkoxy; aryl; and a functional group selected from the group consisting of hydroxyl; thiol; thioether; ketone; aldehyde; ester; ether; amine; imine; amide; nitro; carboxylic acid; disulfide; carbonate; isocyanate; carbodiimide; carboalkoxy; carbamate; and halogen.

14. The metathesis reaction as claimed in claim 13, wherein R is H and R¹ is phenyl or —C═C(CH₃)₂.

15. The metathesis reaction as claimed in any of the preceding claims, wherein L and L¹ are each independently selected from the group consisting of phosphine, sulfonated phosphine, phosphite, phosphinite, phosphonite, arsine, stibine, amine, amide, imine, nitrosyl and pyridine.

16. The metathesis reaction as claimed in any of the preceding claims, wherein each of L and L¹ comprises a compound containing phosphorus.

17. The metathesis reaction as claimed in claim 16, wherein the catalyst of formula I is a compound of formula (II): 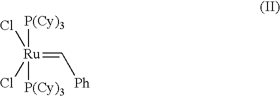
wherein Cy is cyclohexyl.

18. The metathesis reaction as claimed in any of the preceding claims, wherein the phenolic compound comprises a phenol.

19. The metathesis reaction as claimed in claim 18, wherein the phenolic compound comprises phenol.

20. The metathesis reaction as claimed in any of claims 1 to 17, wherein the phenolic compound comprises a substituted phenol which substituted phenol includes at least one hydroxyl and at least one further moiety other than H and OH attached to an arene ring.

21. The metathesis reaction as claimed in any of claims 1 to 17, wherein the phenolic compound comprises an optionally substituted polyaromatic phenol.

22. The metathesis reaction as claimed in any of the preceding claims, wherein the molar ratio of phenolic compound to catalyst is from 1 to 5000 molar equivalents of phenolic compound to ruthenium or osmium.

23. The use of a phenolic compound in the form of phenol or a substituted phenol which substituted phenol includes at least one hydroxyl and a further moiety other than H and OH attached to an arene ring, in a metathesis reaction between at least two olefinic compounds which are the same or different, each olefinic compound comprising a non-cyclic olefin or a compound which includes a non-cyclic olefinic moiety, and the metathesis reaction being carried out in the presence of a catalyst of formula (I) as defined in claim 1.

24. The use of a phenolic compound as claimed in claim 23, to enhance a metathesis reaction, the enhancement being selected from:
i) an increase in lifetime of the catalyst;
ii) an increase in the resistance of the catalyst to olefin feed impurities;
iii) an increase in the selectivity of the metathesis reaction in respect of at least one of the following aspects:
a. reducing the isomerisation of a starting olefinic compound;
b. reducing the formation of secondary metathesis products; and
iv) an increase in the yield of the metathesis product(s);
v) an increase in the rate of reaction; and
vi) the use of lower catalyst concentrations.

25. A product produced by the reaction of any one of claims 1 to 22.
